(19) United States
(12) Patent Application Publication
(10) Pub. No.: US 2024/0173237 A1
(43) Pub. Date: May 30, 2024

(54) COSMETIC COMPOSITION COMPRISING FIBROBLAST GROWTH FACTOR 17

(71) Applicant: Samsung Life Public Welfare Foundation, Seoul (KR)

(72) Inventors: Dong Ik Kim, Seoul (KR); Ae Kyeong Kim, Seoul (KR); Hye Ran Jeon, Chungcheongbuk-do (KR)

(21) Appl. No.: 17/640,755

(22) PCT Filed: Sep. 3, 2020
(86) PCT No.: PCT/KR2020/011832

§ 371 Date: Mar. 4, 2022

(30) Foreign Application Priority Data
Sep. 6, 2019 (KR) 10-2019-0110531
Sep. 1, 2020 (KR) 10-2020-0111297

(51) Int. Cl. A61K 8/64; A61Q 19/08

(52) U.S. Cl. CPC A61K 8/64; A61Q 19/08; A61K 2800/782

(57) ABSTRACT

The present invention relates to a cosmetic composition including fibroblast growth factor 17 (FGF 17). When using the cosmetic composition for skin regeneration including fibroblast growth factor 17 (FGF 17) of the present invention, the proliferation ability of fibroblasts in the skin is promoted such that there is an effect of skin regeneration, synthesis of collagen and elastin is promoted such that it is possible to ameliorate skin wrinkles and improve skin elasticity, and the proliferation ability of aged skin fibroblasts is restored as much as that of unaged skin fibroblasts such that skin aging can be prevented, and therefore, the cosmetic compound can be widely used in the cosmetic field and the pharmaceutical industry.

### TECHNICAL FIELD

The present invention relates to a cosmetic composition containing fibroblast growth factor 17 (FGF 17).

### BACKGROUND ART

Skin aging is largely divided into two types for research. One is ‘internal aging,’ which is an aging phenomenon caused by increasing age, and the other is ‘external aging,’ which is an aging phenomenon caused by external environments such as UV rays or pollution. Several theories have been proposed about skin aging so far, but the most scientifically approached theory on skin aging is the skin aging theory by oxidation. Human skin is composed of the epidermis, dermis, and connective tissue including the stratum corneum, among which the stratum corneum is composed of a dead cell layer formed through a differentiation process of keratinocytes, the basal cells of the epidermis, and plays a role in protecting the human body from the influence of external environments. In addition, the dermal layer, which exists inside the skin, is composed of collagen and elastin, and plays a role in protecting the skin from sagging by giving the skin elasticity. Antioxidant theory is the theory that collagen and elastin are damaged by free radicals generated by external factors such as ultraviolet rays, and accordingly, the elasticity of the skin is reduced, and wrinkles are created.

Collagen is a major matrix protein produced by fibroblasts of the skin and is present in the extracellular matrix, and as its important functions, the mechanical strength of the skin, the resistance of connective tissue and the binding force of tissues, support of cell adhesion, cell division and differentiation induction (during the growth of organisms or wound healing) are known. Such collagen is reduced by age and photoaging by UV irradiation, which is known to be closely related to the formation of wrinkles in the skin. In addition, as extensive research on skin aging has been developed in recent years, an important function of collagen in the skin has been revealed. There are known active ingredients that promote collagen synthesis and show wrinkle alleviation effects. For example, retinoic acid, transforming growth factor (TGF), protein derived from animal placenta, betulinic acid, chlorella extract, etc. are known as collagen synthesis promoting substances. However, when the active ingredients are applied to the skin, there is a limitation in the amount used due to safety issues such as irritation and redness, or the effect of improving the skin function by substantially promoting collagen synthesis in the skin cannot be expected because the effect is insignificant.

Accordingly, there is a need for the development of a substance that effectively promotes skin cell proliferation ability, collagen and elastin synthesis, and has an anti-aging effect while having little or no side effects in vivo.

### DISCLOSURE

### Technical Problem

While studying a substance that may be used as a cosmetic composition, which, as a result, shows a positive effect on skin fibroblasts with few side effects in vivo, the present inventors identified that FGF 17, a type of fibroblast growth factor, promotes the proliferation ability and migration ability for fibroblasts, promotes the synthesis of collagen and elastin, and has an anti-aging effect on fibroblasts, and then completed the present invention.

Accordingly, an aspect of the present invention is to provide a cosmetic composition containing FGF 17 as an active ingredient.

### Technical Solution

One aspect of the present invention provides a cosmetic composition for skin regeneration containing FGF 17.

In addition, the present invention provides a cosmetic composition for preventing skin aging containing FGF 17.

In addition, the present invention provides a cosmetic method for skin regeneration, in which the method includes applying a composition containing FGF 17 to the skin.

In addition, the present invention provides a method for preventing skin aging or regenerating the skin, in which the method includes applying a composition containing FGF 17 to the skin.

### Advantageous Effects

When using the cosmetic composition for skin regeneration containing FGF 17 of the present invention, the proliferation ability of fibroblasts in the skin is promoted such that there is an effect of skin regeneration, synthesis of collagen and elastin is promoted such that it is possible to ameliorate skin wrinkles and improve skin elasticity, and the proliferation ability of aged skin fibroblasts is restored as much as that of unaged skin fibroblasts such that skin aging can be prevented, and therefore, the cosmetic compound can be widely used in the cosmetic field and the pharmaceutical industry.

### DESCRIPTION OF DRAWINGS

FIG. 1 is a diagram identifying changes in proliferation ability, growth rate, and doubling time according to passage of fibroblasts.

FIG. 2 is a diagram measuring the fibroblast proliferation ability of FGF 17 treated by concentration at an early stage of passage of fibroblasts (passages 8 to 14).

FIG. 3 is a diagram measuring the fibroblast migration ability of FGF 17 treated by concentration at an early stage of passage of fibroblasts (passages 8 to 14).

FIG. 4 is a diagram measuring the expression of collagen I of FGF 17 treated by concentration at an early stage of passage of fibroblasts (passages 8 to 14).

FIG. 5 is a diagram measuring the expression of MMP1 of FGF 17 treated by concentration at an early stage of passage of fibroblasts (passages 8 to 14).

FIG. 6 is a diagram comparing the proliferation ability of fibroblasts treated with FGF 17 at a late stage of passage with the proliferation ability of fibroblasts at an early stage of passage.

FIG. 7 is a diagram illustrating a comparison of the expression of collagen I gene, collagen III gene, and elastin gene according to the treatment of FGF 17 in fibroblasts at an early stage of passage (passage 9) and fibroblasts at a late stage of passage (passage 22). (p9: passage 9, p22: passage 22)

FIG. 8 is a diagram identifying the expression of elastin in the experimental group treated with FGF 17 100 ng/ml in fibroblasts at a late stage of passage.

FIG. 9 is a diagram illustrating an average doubling time of fibroblasts at an early stage of passage (passages 5 to 11) and fibroblasts at a late stage of passage (passages 21 to 26).

FIG. 10 is a diagram illustrating changes in doubling time when FGF 17 is treated with fibroblasts at an early stage of passage (passage 11) and fibroblasts at a late stage of passage (passage 26).

FIG. 11 is a diagram illustrating changes in doubling time when FGF 17 is treated once or twice with fibroblasts at a late stage of passage (passage 26).

FIG. 12 is a diagram illustrating the effect of the FGF family on the proliferation of fibroblasts at an early stage of passage (passage 11).

FIG. 13 is a diagram illustrating the effect of the FGF family on the proliferation of fibroblasts at a late stage of passage (passage 26).

FIG. 14 is a diagram identifying the proliferation restoration ability when oxidative aging-induced fibroblasts are treated with FGF 17 at various concentrations.

FIG. 15 is a diagram illustrating changes in the expression of skin elasticity-related genes in fibroblasts by FGF 17 treatment.

FIG. 16 is a diagram illustrating changes in the expression of skin regeneration or elasticity-related proteins in fibroblasts by FGF 17 treatment.

### MODES OF THE INVENTION

In order to achieve the object of the present invention, one aspect of the present invention provides a cosmetic composition for skin regeneration containing FGF 17.

Hereinafter, the present invention will be described in more detail. FGF 17 of the present invention is a protein encoded by the human FGF 17 gene, one of fibroblast growth factors. It is well known in the pertinent field that fibroblast growth factors are involved in a variety of biological processes including embryonic developmental cell growth, morphogenesis, tissue repair, tumor growth and invasion. Among them, FGF 17 is known as a protein necessary for normal brain development by playing an important role in the regulation of embryonic development and patterning of the embryonic brain. In mice, FGF 17 is known to play an important role in the central nervous system, bone and blood vessel growth by developing specific regions of the brain, skeleton, and arteries. The FGF 17 may be purchased and used in the usual way or FGF 17 derived from stem cells cultured under hypoxic culture conditions may be used but is not limited thereto.

The FGF 17 of the present invention may promote the proliferation ability of cells, preferably, it may promote the proliferation ability of fibroblasts. In addition, when the FGF 17 of the present invention is treated, the proliferation ability of aged fibroblasts may be enhanced to an extent equivalent to that of unaged fibroblasts, and preferably, the proliferation ability of fibroblasts passaged from 21 to 26 times may be enhanced as much as the proliferation ability of fibroblasts passaged from 5 to 14 times. The FGF 17 of the present invention may be included in various concentrations and may increase the proliferation ability of aged cells to the level of proliferation ability of unaged cells or higher. Accordingly, the composition containing FGF 17 of the present invention may be for promoting proliferation ability of fibroblasts or for skin regeneration.

The FGF 17 of the present invention may promote the migration ability of cells, preferably promote the migration ability of fibroblasts. Accordingly, the composition containing FGF 17 of the present invention may be for promoting the migration ability of fibroblasts.

The FGF 17 of the present invention may promote the expression of collagen 1 and collagen 3 in cells and decrease the expression of MMP1 and elastase, and preferably promote the expression of collagen 1 and collagen 3 in fibroblasts and decrease the expression of MMP1 and elastase. Accordingly, the composition containing FGF 17 of the present invention may be for promoting collagen expression and for decreasing expressions of MMP1 and elastase.

The FGF 17 of the present invention may have excellent ability to restore aged fibroblasts to the state of unaged fibroblasts, and preferably restore the proliferation ability of aged fibroblasts to the level of proliferation ability of unaged fibroblasts and may be to enhance the expression of collagen I gene, collagen III gene, elastin gene in aged fibroblasts, and to decrease the expression of elastase. The composition containing FGF 17 of the present invention may contain 10 to 2,000 ng/ml of FGF 17, preferably 30 to 500 ng/ml.

In the present invention, the term “skin regeneration” may refer to any action that replenishes a part of the skin or promotes the proliferation of skin cells when a part of the skin is lost. When the proliferation of fibroblasts is promoted, the skin regeneration effect may appear. The FGF 17 of the present invention has an excellent proliferation promoting effect on fibroblasts and has an excellent skin regeneration effect.

In the present invention, the term “cosmetic composition” is a composition containing the FGF 17, and the formulation may be in any form. For example, cosmetics prepared using the cosmetic composition are creams, packs, lotions, essences, face lotions, foundations, makeup bases, and the like, and in order to achieve the object of the present invention, it may be manufactured and commercialized in any form of these formulations but is not limited to the above examples.

In the present invention, the cosmetic composition may further include one or more additives selected from the group consisting of purified water, polyhydric alcohol, surfactant, viscosity modifier, chelating agent, emulsifier, pH adjuster, acid-alkali agent, antioxidant, humectant, brightener, preservative, flavoring agent, fragrance, gelling agent, stabilizer, colorant, and pigment.

In addition, the present invention provides a cosmetic composition for preventing skin aging containing FGF 17.

The FGF 17 of the present invention may be included in various concentrations, and in particular, it may increase the proliferation ability of aged cells to the level of proliferation ability of unaged cells or higher, thereby having an excellent ability to restore aged fibroblasts to the state of unaged fibroblasts, and it may increase the expression of collagen and elastin and decrease the expression of elastase to restore skin elasticity to a pre-aging state. Accordingly, the composition containing FGF 17 of the present invention may be for preventing skin aging.

In the present invention, the term “aging” is caused by the passage of time, external environments, etc., and shows symptoms such as reduction in elasticity of the skin, reduction in shine, generation of wrinkles, decreased regeneration, or severe drying. Accordingly, the “prevention of skin aging” of the present invention may be any one or more uses selected from the group consisting of wrinkle alleviation, skin elasticity enhancement, skin regeneration enhancement, and skin shine improvement.

In the present invention, the composition may promote the expression of collagen by FGF17 or decrease the expression of MMP1.

In the present invention, the term “wrinkle alleviation” refers to maintaining or enhancing the skin's ability related to wrinkle and elasticity. Among the structures of the skin, collagen fibers (collagen) and elastic fibers (elastin) present in the dermis layer are the main proteins that play the role. They control skin elasticity, and the biosynthesis of collagen is affected internally and externally by the skin. The FGF17 of the present invention has excellent collagen synthesis ability and elastin synthesis ability and has an effect of decreasing the expression of elastase, an enzyme that decomposes elastin, and thus has an excellent wrinkle alleviation effect. Accordingly, the composition containing FGF 17 of the present invention may be for alleviating wrinkles and enhancing skin elasticity.

In addition, the present invention provides a cosmetic method for skin regeneration, in which the method includes applying a composition containing FGF 17 to the skin.

In the present invention, the application of the composition containing FGF 17 to the skin may be applying the composition to the skin one or more times, preferably applying the composition two or more times, more preferably applying the composition two or more times, at intervals of 16 to 24 hours. When the composition is applied two or more times, the doubling time of skin cells is shortened compared to the case of applying the composition once, which may have an effect of helping skin regeneration.

In addition, the present invention provides a method for preventing skin aging or regenerating the skin, in which the method includes applying a composition containing FGF 17 to the skin.

Terms not otherwise defined in the present specification have the meanings commonly used in the technical field to which the present invention pertains.

Hereinafter, the present invention is described in detail by way of the examples. However, the following examples only illustrate the present invention, and the content of the present invention is not limited by the following examples.

### Example 1. Identification of Promotion of Proliferation Ability of Fibroblasts of FGF 17

### 1.1. Identification of Changes in Proliferation Ability, Growth Rate, and Doubling Time According to Passage of Fibroblasts

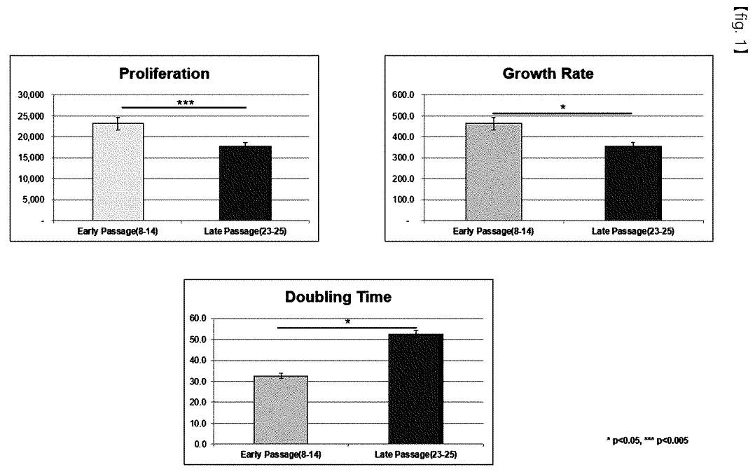

An experiment was performed to identify changes in proliferation ability, growth rate, and doubling time according to passage of fibroblasts. Specifically, fibroblasts were cultured using fibroblast growth medium 2 (PromoCell), and 5,000 cells per 1 cm² were seeded. The medium was changed once every 2-3 days, and by measuring the number of cells proliferated by passage, proliferation ability, growth rate, and doubling time were identified. The proliferation ability measured at passages 8-14 and 23-25 is shown in Table 1, the growth rate is shown in Table 2, and the doubling time is shown in Table 3. In addition, a graph showing the same is shown in FIG. 1.

TABLE 1

| | |
|---|---|
| Passages 8 to 14 | Passages 23 to 25 |

| | | | | |
|---|---|---|---|---|
| Proliferation ability | 23,209 | cell/cm² | 17,753 | cell/cm² |
| Error | 1,498 | cell/cm² | 845 | cell/cm² |

| | |
|---|---|
| P value | 0.02674 |

TABLE 2

| | Passages 8 to 14 | Passages 23 to 25 |
|---|---|---|
| Growth rate (%) | 464.2 | 355.1 |
| Error | 30.0 | 16.9 |

| | |
|---|---|
| P value | 0.02674 |

TABLE 3

| | Passages 8 to 14 | Passages 23 to 25 |
|---|---|---|
| Doubling time | 32.7 hours | 52.7 hours |
| Error | 1.2 | 1.7 |

| | |
|---|---|
| P value | 0.0033 |

As shown in Tables 1 to 3 and FIG. 1, it was identified that the proliferation rate and growth rate of fibroblasts decreased as the passage progressed, whereas the doubling time increased.

### 1.2. Identification of Effects of Promoting Proliferation Ability on Fibroblasts of FGF 17

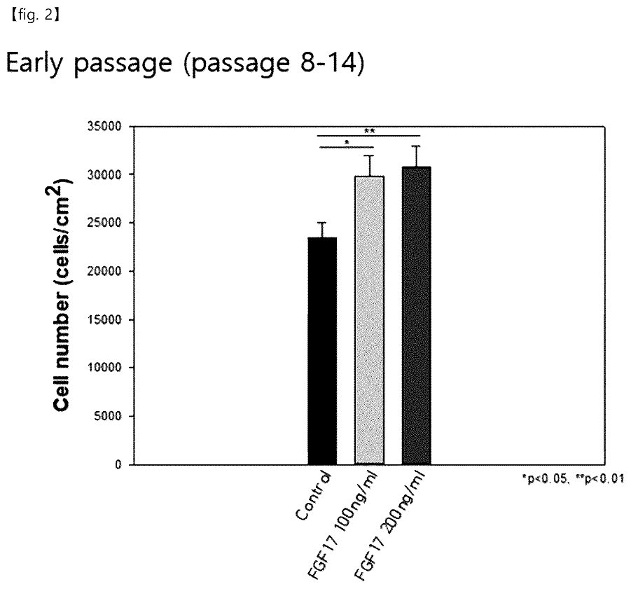

An experiment was performed to identify whether FGF 17 had an effect of promoting a proliferation ability on fibroblasts. The control group was treated with only fibroblast growth medium 2, and the experimental group was treated with fibroblasts at an early stage of passage of fibroblasts (passages 8 to 14) by varying the concentration of FGF 17 from 100 ng/ml to 500 ng/ml, and the number of cells was measured to identify an effect of promoting a proliferation ability of fibroblasts according to the treatment concentration of FGF 17. FGF 17 was treated with fibroblasts at an early stage of passage (passages 8 to 14), and the measured values for the number of cells are shown in FIG. 2 and Table 4.

TABLE 4

| Group | Cell/cm² |
|---|---|
| Control group | 23,412 |
| FGF 17 100 ng/ml | 29,796 |
| FGF 17 200 ng/ml | 30,801 |

As shown in FIG. 2 and Table 4, as a result of treatment with each concentration of FGF 17 at an early stage of passage, it was identified that the proliferation ability of fibroblasts increased the most when 100 and 200 ng/ml were treated.

### Example 2. Identification of Promotion of Migration Ability for Fibroblasts of FGF 17

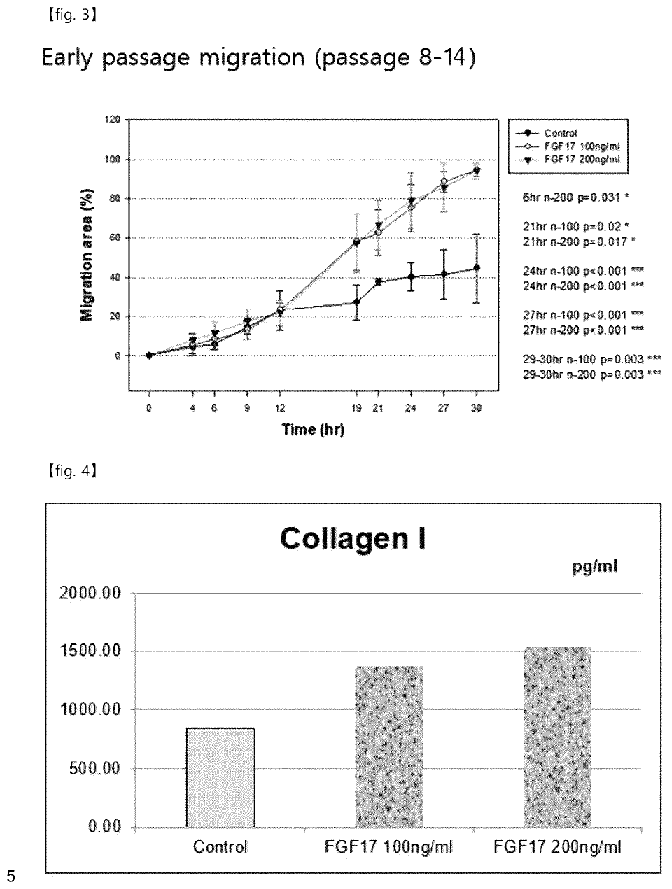

An experiment was performed to identify the effect of promoting the migration ability of FGF 17 on fibroblasts. Specifically, fibroblasts were cultured using fibroblast growth medium 2 (PromoCell), and 5,000 cells per 1 cm² were seeded. The medium was changed once every 2-3 days and passaged. The control group was treated with only fibroblast growth medium 2, and the experimental group was treated with 100 or 200 ng/ml of FGF 17 at an early stage of passage (passages 8 to 14). The migration ability of fibroblasts was measured using a 24-well cell migration assay kit (Cell biolabs, INC.) for 30 hours, which is shown in FIG. 3.

As shown in FIG. 3, the migration ability of the control group did not exceed 50%, but in the case of the FGF 17 treatment group, it was identified that the migration ability exceeded 90% in either the 100 or 200 ng/ml treatment group, indicating that FGF 17 promoted the migration ability of fibroblasts.

### Example 3. Identification of Effects of Collagen I and MMP1 Expression on Fibroblasts of FGF 17

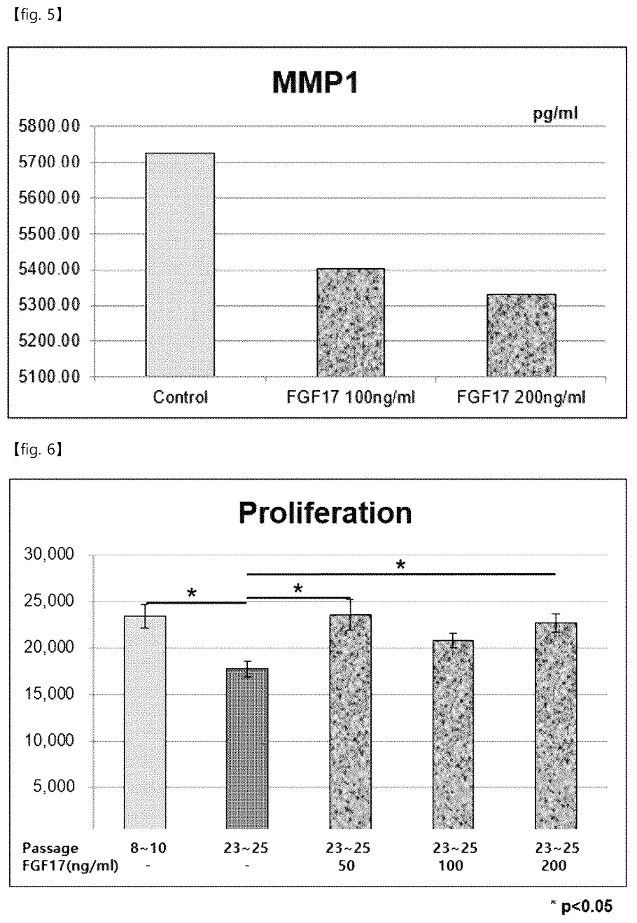

An experiment was performed to identify the effect of FGF 17 on the expression of collagen I, an elasticity improving factor, and MMP1, an elasticity inhibitory factor, on fibroblasts. Specifically, fibroblasts were cultured using fibroblast growth medium 2 (PromoCell), and 5,000 cells per 1 cm² were seeded. The medium was changed once every 2-3 days and passaged. The control group was treated with only fibroblast growth medium 2, and the experimental group was treated with 100 or 200 ng/ml of FGF 17 at an early stage of passage (passages 8 to 14). The expression of MMP1, an elasticity inhibitory factor, and collagen I in fibroblasts was measured using the ELISA kit Human pro-collagen I alpha1 and Human pro-MMP-1 (R&D Systems). The expression of collagen I is shown in FIGS. 4 and 5, and the expression of MMP1 is shown in FIG. 5 and Table 6.

TABLE 5

| | Control group | FGF 17 100 ng/ml | FGF 17 200 ng/ml |
|---|---|---|---|
| Unit pg/ml | 839.79 | 1366.94 | 1532.54 |

TABLE 6

| | Control group | FGF 17 100 ng/ml | FGF 17 200 ng/ml |
|---|---|---|---|
| Unit pg/ml | 5725.99 | 5404.11 | 5332.10 |

As shown in FIGS. 4 and 5, it was identified that the expression of collagen I was increased by the treatment of FGF 17, and it was identified that the expression was higher in the 200 ng/ml treatment than in the 100 ng/ml treatment, thus identifying that the expression was increased in a concentration-dependent manner.

As shown in FIG. 5 and Table 6, it was identified that the expression of MMP1 was decreased by the treatment of FGF 17, and it was identified that the expression was significantly decreased in the 200 ng/ml treatment than in the 100 ng/ml treatment, thus identifying that the expression was decreased in a concentration-dependent manner.

### Example 4. Identification of Reverse Aging Effect on Fibroblasts of FGF

### 4.1. Identification of Promotion of Proliferation Ability for Fibroblasts of FGF 17 at Late Stage of Passage

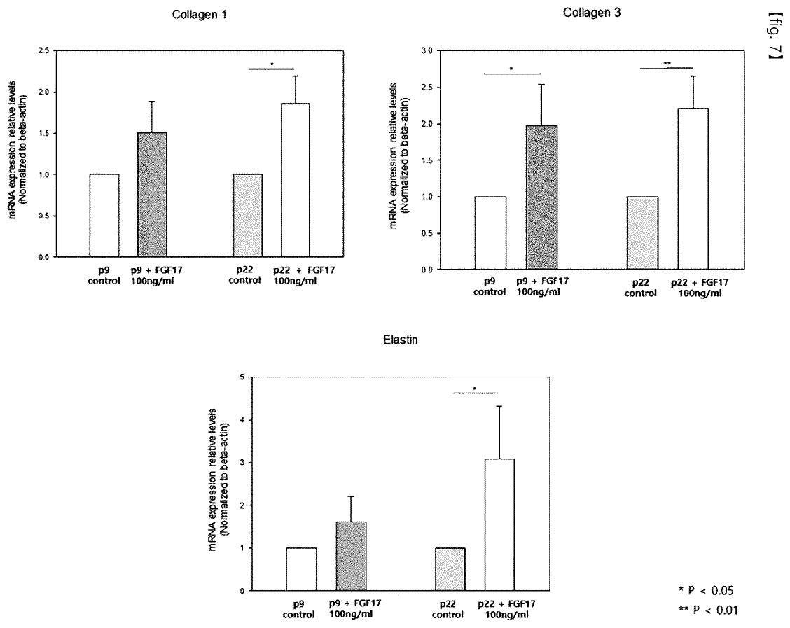

In order to identify whether FGF 17 has a reverse aging effect, an experiment was performed to compare the proliferation ability when FGF 17 was treated with fibroblasts at a late stage of passage with the proliferation ability of the fibroblasts at an early stage of passage. Specifically, fibroblasts were cultured using fibroblast growth medium 2 (PromoCell), and 5,000 cells per 1 cm² were seeded. The medium was changed once every 2-3 days and passaged. The proliferation abilities of the control group treated only with fibroblast growth medium 2 at an early stage of passage, the control group treated only with fibroblast growth medium 2 at a late stage of passage, the group treated with FGF 17 50 ng/ml at a late stage of passage, the group treated with FGF 17 100 ng/ml at a late stage of passage, and the group treated with FGF 17 200 ng/ml at a late stage of passage were compared by measuring the number of cells. The results of measuring the number of cells in the experimental group are shown in FIG. 6 and Table 7.

TABLE 7

| Group | Cell/cm² |
|---|---|
| Early stage of passage (passages 8 to 14) | 23,412 |
| Late stage of passage (passages 23 to 25) | 17,765 |
| Late stage of passage + FGF 17 50 ng/ml | 23,531 |
| Late stage of passage + FGF 17 100 ng/ml | 20,759 |
| Late stage of passage + FGF 17 200 ng/ml | 22,667 |

As shown in FIG. 6, when FGF 17 was treated with fibroblasts at a late stage of passage, it was identified that the proliferation ability was increased compared to the control group in which FGF 17 was not treated with cells at a late stage of passage. In addition, it was identified that the increase in proliferation ability was the greatest in the experimental group treated with FGF 17 at 50 ng/ml, and increased to a level exceeding the proliferation ability of fibroblasts at an early stage of passage. Accordingly, the reverse aging effect of FGF 17 was identified by identifying that FGF 17 increased the proliferation ability of aged cells to the level of proliferation ability of unaged cells through successive passages.

### 4.2. Identification of Promotion of Proliferation Ability for Fibroblasts of FGF 17 at Late Stage of Passage

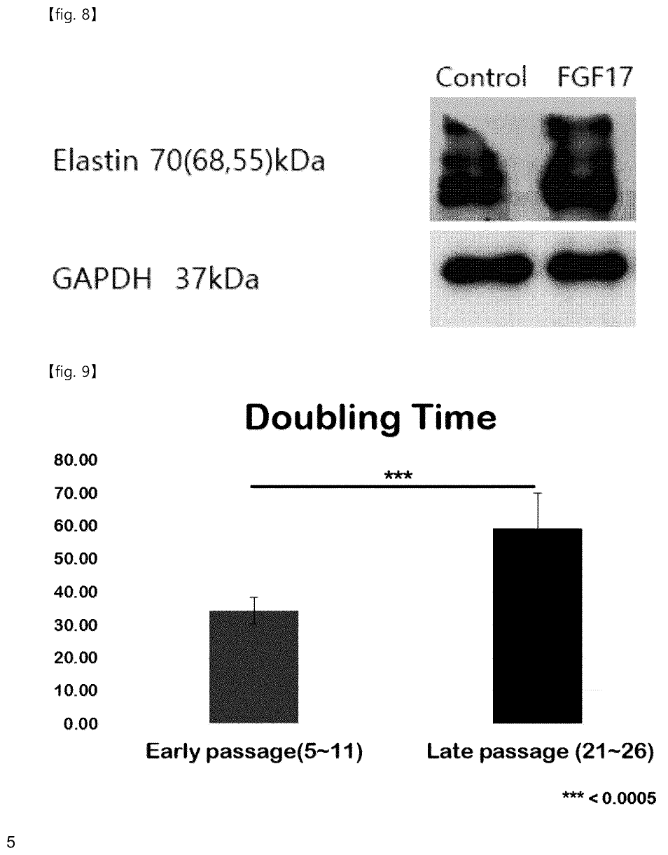

In order to identify whether FGF 17 has a reverse aging effect, an experiment was performed to compare the expression of collagen I gene, collagen III gene, and elastin gene when FGF 17 was untreated and FGF 17 100 ng/ml was treated to fibroblasts at an early stage of passage (passage 9) and fibroblasts at a late stage of passage (passage 22). Specifically, fibroblasts were cultured using fibroblast growth medium 2 (PromoCell), and 5,000 cells per 1 cm² were seeded. The medium was changed once every 2-3 days and passaged. The gene expression levels were compared by amplifying the genes by PCR using the primers shown in Table 8. The nucleotide sequences of the primers used for PCR are shown in Table 8, and the measurement results of gene expression levels are shown in FIG. 7.

Collagen I, collagen III, and elastin expressions in the experimental group treated with FGF 17 100 ng/ml with fibroblasts at a late stage of passage were analyzed by qRT-PCR, and the elastin expression results are shown in FIG. 8.

TABLE 8

| Primers | Nucleotide sequences (5′→3′) |
|---|---|
| beta-Actin (F) | AGTCCTGTGGCATCCACGAA (SEQ ID: 1) |
| beta-Actin (R) | GATCCACACGGAGTACTTGC (SEQ ID: 2) |
| Collagen I (F) | CCCTCAAGGTTTCCAAGGAC (SEQ ID: 3) |
| Collagen I (R) | ACCAGGTTCACCCTTCACAC (SEQ ID: 4) |
| Collagen III (F) | TGAAAGGACACAGAGGCTTCG |
| | (SEQ ID: 5) |
| Collagen III (R) | GCACCATTCTTACCAGGCTC (SEQ ID: 6) |
| Elastin (F) | CTGCAAAGGCAGCCAAATAC (SEQ ID: 7) |
| Elastin (R) | CACCAGGAACTAACCCAAACT |
| | (SEQ ID: 8) |

As shown in FIG. 7, it was identified that the expression levels of the collagen I gene, the collagen III gene, and the elastin gene all increased compared to the control group when FGF 17 100 ng/ml was treated at an early stage of passage (passage 9). It was identified that the expression levels of the collagen I gene, the collagen III gene, and the elastin gene all increased when FGF 17 100 ng/ml was treated at a late stage of passage (passage 22). In particular, it was identified that the increase in the expressions of collagen I gene, collagen III gene, and elastin gene according to FGF 17 treatment was higher in fibroblasts at a late stage of passage (passage 22) than in fibroblasts at an early stage of passage (passage 9). The reverse aging effect of FGF 17 was identified once again.

As shown in FIG. 8, it was identified that elastin was highly expressed in the experimental group treated with FGF 17 100 ng/ml in fibroblasts at a late stage of passage.

### Example 5. Identification of Effects of FGF 17 on Fibroblast Doubling Time

### 5.1. Identification of Changes in Doubling Time According to Passage of Fibroblasts

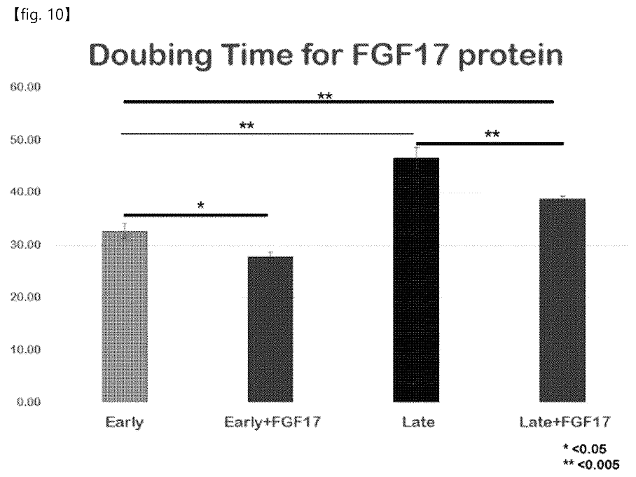

In order to verify the effect according to the number of times of treatment and treatment time of FGF 17 with fibroblasts, first, an experiment was performed to identify the change in the doubling time according to the passage of fibroblasts. Specifically, the experiment was performed in the same manner as the experiment to identify the change in doubling time of Example 1.1, except that the experimental group at an early stage of passage was passages 5 to 11, and the experimental group at a late stage of passage was passages 21 to 26. The change in doubling time is shown in FIG. 9.

As shown in FIG. 9, the average doubling time of fibroblasts was identified to be 34.23 hours at an early stage of passage (passages 5 to 11), and 59.36 hours at a late stage of passage (passages 21 to 26). As the passage progressed, it was confirmed that the doubling time increased.

### 5.2. Identification of Effects of FGF 17 on Doubling Time of Fibroblasts According to Fibroblast Passage Period

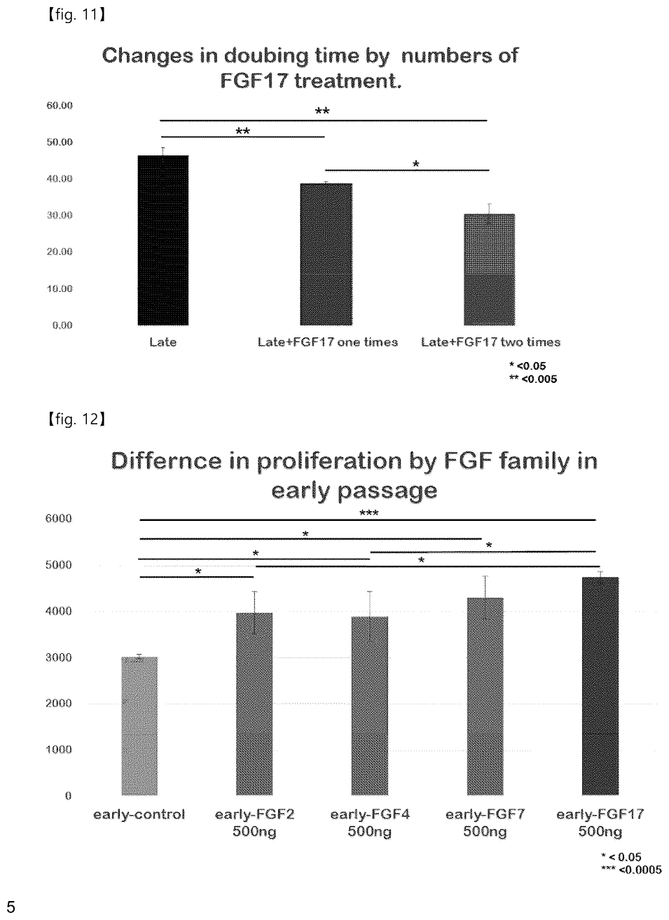

An experiment was performed to identify the effect of FGF 17 on the doubling time of fibroblasts according to the passage period of fibroblasts. Fibroblasts were passaged, and 500 ng/ml of FGF 17 was treated at an early stage of passage (passage 11) and at a late stage of passage (passage 26), and the doubling time was measured. The measurement results are shown in FIG. 10.

As shown in FIG. 10, it was identified that after both the fibroblasts at an early stage of passage (passage 11) and at a late stage of passage (passage 26) were treated with FGF 17, the doubling time was reduced.

### 5.3. Identification of Effect on Reduction of Doubling Time of Fibroblasts According to Number of FGF 17 Treatments

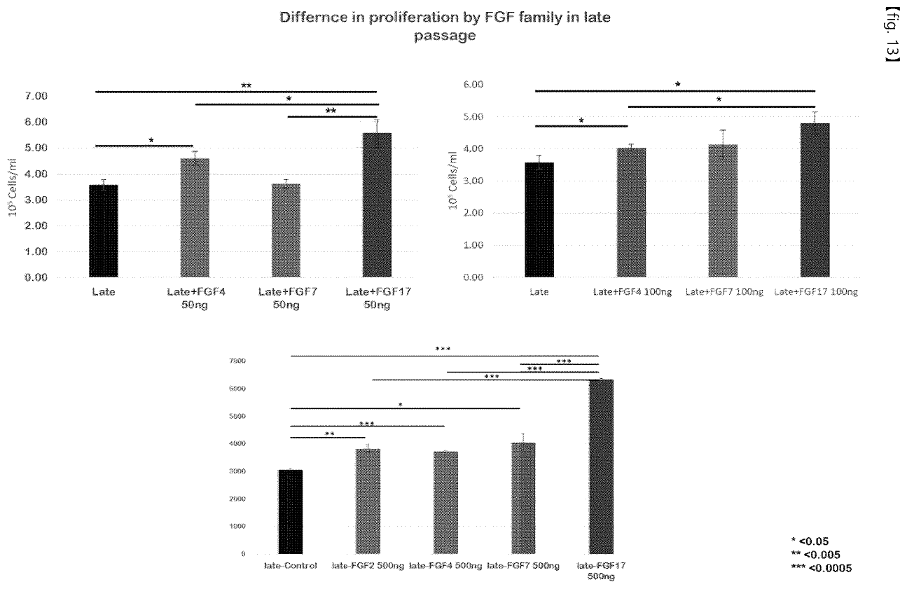

An experiment was performed to identify the effect on the reduction of the doubling time of fibroblasts according to the number of FGF 17 treatments. Specifically, fibroblasts at a late stage of passage (passage 26) were seeded on the plate. The ‘control group’ that is not treated with anything and fibroblasts at a late stage of passage (passage 26) were seeded on the plate. After 4 hours of seeding, fibroblasts at a late stage of passage (passage 26) and a ‘one-time experimental group treated with 500 ng/ml of FGF 17 were seeded on the plate. After 4 hours of seeding, 500 ng/ml of FGF 17 was treated once, and after 24 hours of seeding, it was washed once with PBS, and a ‘two-time experimental group’ additionally treated with 500 ng/ml of FGF 17 once was set up. Thereafter, the doubling time of the experimental groups and the control group was measured, and the measurement results are shown in FIG. 11.

As shown in FIG. 11, it was identified that the ‘two-time experimental group,’ in which FGF 17 was treated twice at 20-hour intervals, had a significantly greater reduction in doubling time than the ‘one-time experimental group.’

From these results, it was identified that FGF 17 reduced the doubling time of passaged cells after being treated with fibroblasts. In addition, when treated several times at regular time intervals, it was identified that the doubling time was further significantly reduced, thereby increasing the proliferation rate of fibroblasts.

### Example 6. Effect of FGF Family on Proliferation of Fibroblasts

### 6.1. Effect of FGF Family on Fibroblasts at Early Stage of Passage

Among the FGF families, the effects of FGF 2, FGF 4, FGF 7 and FGF 17, which are FGF families involved in the proliferation of fibroblasts, on the proliferation of fibroblasts at an early stage of passage were compared. Specifically, 5,000 cells/cm² of fibroblasts were seeded and passaged in the same manner as in Example 1.1. Thereafter, fibroblasts at an early stage of passage (passage 11) were seeded at 10,000 cells/cm². After about 24 hours of seeding, FGF 2, FGF 4, FGF 7 and FGF 17 were treated at a concentration of 500 ng/ml, respectively, and cell proliferation was measured in the same manner as in Example 1.1. The results are shown in FIG. 12.

As shown in FIG. 12, it was identified that the proliferation of fibroblasts was significantly increased in all of the FGF 2, FGF 4, FGF 7 and FGF 17 treatment groups compared to the control group. In particular, it was identified that the FGF 17 treatment group promoted the proliferation of fibroblasts to a significant extent compared to other FGF family groups.

### 6.2. Effect of FGF Family on Fibroblasts at Late Stage of Passage

Among the FGF families, the effects of FGF 4, FGF 7, and FGF 17, which are FGF families involved in the proliferation of fibroblasts, on the proliferation of fibroblasts at a late stage of passage were compared. Specifically, FGF 4, FGF 7, and FGF 17 were treated at a concentration of 50 ng/ml, 100 ng/ml or 500 ng/ml, respectively, with fibroblasts in a late stage of passage (passage 26), and cell proliferation was measured in the same manner as in Example 1.1. The results are shown in FIG. 13.

As shown in FIG. 13, it was identified that the proliferation of fibroblasts was significantly increased in all of the FGF 4, FGF 7 and FGF 17 treatment groups compared to the control group. In addition, the FGF 17 treatment group promoted the proliferation of fibroblasts the most at all concentrations. In particular, it was identified that when treated at a concentration of 50 ng/ml or 500 ng/ml, the FGF 17 treatment group promoted the proliferation of fibroblasts to a significant extent compared to other FGF family groups.

### Example 7. Identification of Proliferation Restoration Ability for Fibroblasts Damaged by Oxidative Stress of FGF 17

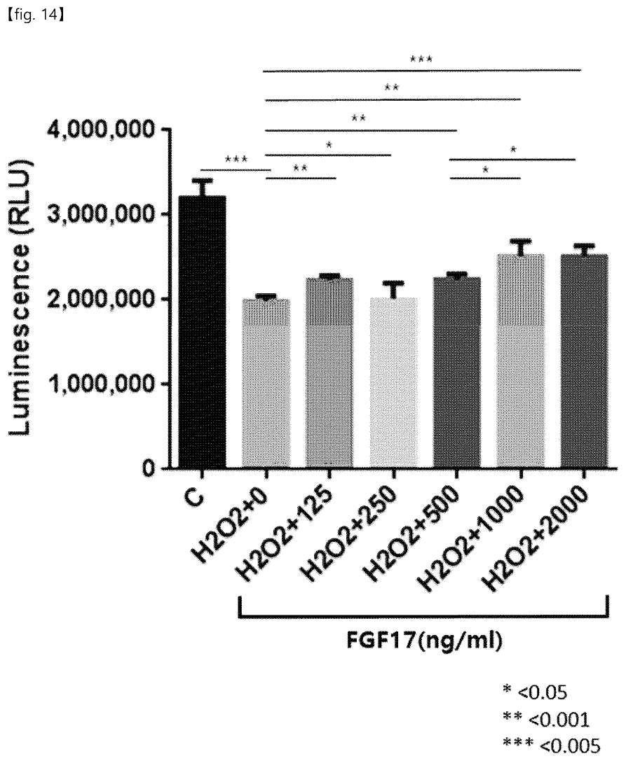

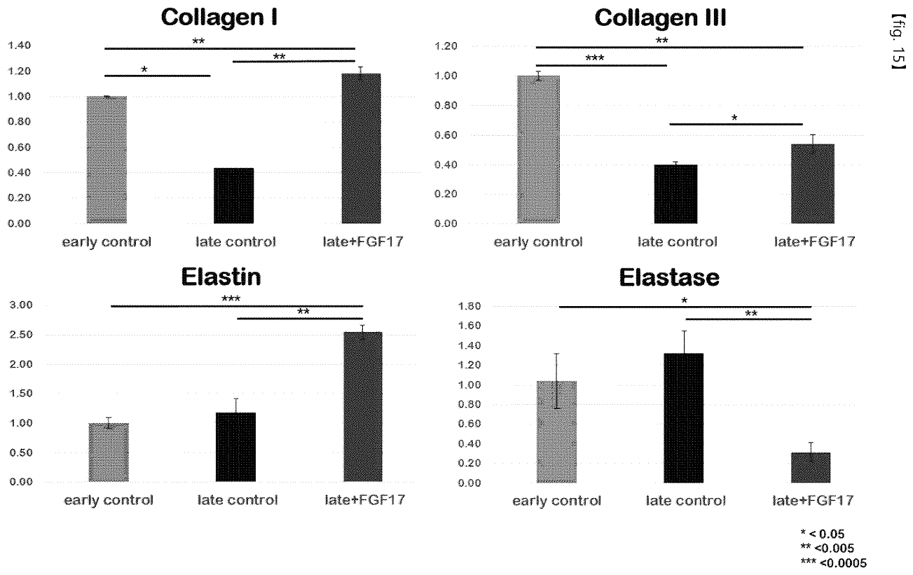

An experiment was performed to identify whether the proliferation ability of fibroblasts was restored when fibroblasts damaged by oxidative stress were treated with FGF 17 at various concentrations. Specifically, fibroblasts of passages 7 to 9 were seeded in 96-well plates at about 2,500 cells/well. After 24 hours of seeding, it was replaced with a low glucose (1 g/L) DMEM medium containing no penicillin, and starvation was performed. Again, after 24 hours, H₂O₂ was diluted to 400 uM in the low glucose (1 g/L) DMEM medium containing no FBS and penicillin, and the cells were treated for 3 hours and 30 minutes to induce artificial oxidative aging. Thereafter, FGF 17 was diluted to 125 ng/ml, 250 ng/ml, 500 ng/ml, 1,000 ng/ml and 2,000 ng/ml in serum-free medium, and was treated with oxidative aging-induced cells for 3 days. The number of cells was measured by a method of measuring luminescence using Promega's CellTiter-Glo® 2.0 cell viability assay reagent. The results are shown in FIG. 14.

As shown in FIG. 14, it was identified that the proliferation ability was restored to a significant extent compared to the experimental group not treated with FGF 17 in the FGF 17-treated experimental group. In particular, in the experimental group treated with FGF 17 at 1,000 ng/ml and 2,000 ng/ml concentrations, it was identified that the proliferation of fibroblasts was remarkably restored.

### Example 8. Identification of Effects of FGF 17 on Expression of Skin Elasticity-Related Genes

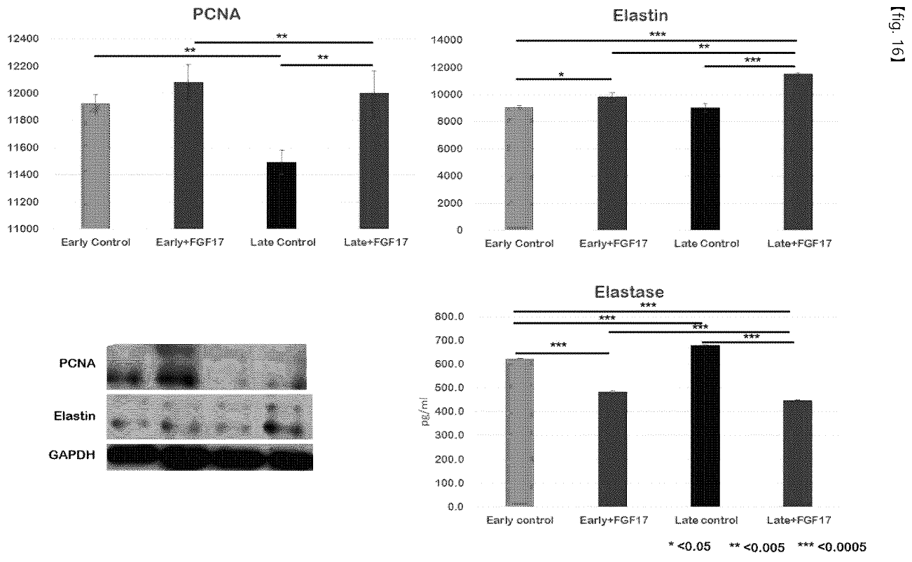

An experiment was performed to identify the effect of FGF 17 on the gene expressions of collagen I, collagen III, elastin, and elastase, which are substances related to skin elasticity. Specifically, a fibroblast growth medium (fibroblast growth medium 2, PromoCell) was used for fibroblasts at an early stage of passage and fibroblasts at a late stage of passage, and 5,000 cells per 1 cm² were seeded and passaged. Fibroblasts at an early stage of passage (passage 11) and at a late stage of passage (passage 26) were seeded at 10,000 cells per 1 cm², and FGF 17 was treated at a concentration of 500 ng/ml after 24 hours. On the second day after treatment, RNA was isolated and the gene expressions of collagen I, collagen III, elastin, and elastase were measured through Qrt-PCR, and the measurement results are shown in FIG. 15. Primers for gene amplification are shown in Table 9.

TABLE 9

| Primers | Nucleotide sequences (5′→3′) |
|---|---|
| GAPDH (F) | GTCGGAGTCAACGGATTTGG (SEQ ID: 9) |
| GAPDH (R) | GGGTGGAATCAATTGGAACAT |
| | (SEQ ID: 10) |
| Collagen I (F) | TGCGATGACGTGATCTGTGA (SEQ ID: 11) |
| Collagen I (R) | TTGGTCGGTGGGTGACTCTG (SEQ ID: 12) |
| Collagen III (F) | TGAAAGGACACAGAGGCTTCG |
| | (SEQ ID: 13) |
| Collagen III (R) | GCACCATTCTTACCAGGCTC (SEQ ID: 14) |
| Elastin (F) | CTGCAAAGGCAGCCAAATAC (SEQ ID: 15) |
| Elastin (R) | CACCAGGAACTAACCCAAACT |
| | (SEQ ID: 16) |
| Elastase (F) | TTCCTCGCCTGTGTCCTG (SEQ ID: 17) |
| Elastase (R) | CTGCAGGGACACCATGAA (SEQ ID: 18) |

As shown in FIG. 15, it was identified that the group treated with FGF 17 significantly increased the expressions of collagen I, collagen III, and elastin compared to the group not treated with FGF 17, and the expression of elastase, an enzyme that decomposes elastin, was decreased. Based on these results, it was identified that FGF 17 has the effect of promoting the expression of factors that help skin elasticity and lowering the expression of factors that inhibit skin elasticity.

### Example 9. Identification of Effects of FGF 17 on Skin Regeneration or Elasticity-Related Protein Expression

A proliferating cell nuclear antigen (PCNA) is a non-histone nuclear protein known to play an important role in causing cell proliferation. In this Example, in order to identify the expression of skin regeneration or elasticity-related protein of FGF 17, the expressions of PCNA, elastin, and elastase were identified. Specifically, the fibroblasts at an early stage of passage and the fibroblasts at a late stage of passage were passaged by seeding 5,000 cells per 1 cm² using fibroblast growth medium 2 (PromoCell), and the fibroblasts at an early stage of passage (passage 11) and a late stage of passage (passage 26) were seeded at 10,000 cells per cm². After 24 hours of seeding, fibroblasts were treated with FGF 17 at a concentration of 500 ng/ml, and proteins were isolated on the second day after FGF 17 treatment. PCNA antibody (proliferating cell nuclear antigen, Dakocytomation, M0879), elastin antibody (abcam, ab9519) and GAPDH antibody (abcam, ab9485) were used to identify the expressions of PCNA and elastin proteins by Western blot. Elastase identified protein expression through ELISA using R&D System's DY9167-05 and DY008 kits, and the protein expression results are shown in FIG. 16.

As shown in FIG. 16, in the case of PCNA, it was identified that the expression level was increased in the group treated with FGF 17 in the fibroblasts at a late stage of passage, and in the case of elastin, it was identified that the expression was increased by FGF 17 treatment both at an early stage and a late stage of passage. In the case of elastase, it was identified that the expression was rapidly decreased by FGF 17 treatment both at an early stage and a late stage of passage.

### Formulation Example 1: Face Lotion

A formulation example of face lotion among cosmetics containing FGF 17 of the present invention is as follows.
Ingredients (content, unit: wt %)
FGF 17 of the present invention 2.0
Glycerin 5.0
1,3-butylene glycol 3.0
PEG 1500 1.0
Allantoin 0.1
DL-panthenol 0.3
EDTA-2Na 0.02
Benzophenone-9 0.04
Sodium hyaluronate 5.0
Ethanol 10.0
Octyldodeceth-16 0.2
Polysorbate 20 0.2
Trace amounts of preservatives, fragrances, and pigments
A balance of distilled water
Total 100

### Formulation Example 2: Cream

A prescription example of cream among cosmetics containing FGF 17 of the present invention is as follows.
Ingredients (content, unit: wt %)
FGF 17 of the present invention 2.0
Lipophilic monostearate glycerin 2.0
Cetearyl alcohol 2.2
Stearic acid 1.5
Beeswax 1.0
Polysorbate 60 1.6
Sorbitan stearate 0.6
Hydrogenated vegetable oil 1.0
Squalane 3.0
Mineral oil 5.0
Trioctanoin 5.0
Dimethicone 1.0
Sodium magnesium silicate 0.1
Glycerin 5.0
Betaine 3.0
Triethanolamine 1.0
Sodium hyaluronate 4.0
Trace amounts of preservatives, fragrances, and pigments
A balance of distilled water
Total 100

### Formulation Example 3: Essence

A prescription example of essence among cosmetics containing FGF 17 of the present invention is as follows.
Ingredients (content, unit: wt %)
FGF 17 of the present invention 2.0
Glycerin 10.0
Betaine 5.0
PEG 1500 2.0
Allantoin 0.1
DL-panthenol 0.3
EDTA-2Na 0.02
Benzophenone-9 0.04
Hydroxyethyl cellulose 0.1
Sodium hyaluronate 8.0
Carboxyvinyl polymer 0.2
Triethanolamine 0.18
Octyldodecanol 0.3
Octyldodeceth-16 0.4
Ethanol 6.0
Trace amounts of preservatives, fragrances, and pigments
A balance of distilled water
Total 100

As discussed above, the specific portions of the contents of the present invention have been described in detail. Therefore, it is apparent to a person having ordinary skill in the pertinent art that such specific technology is merely a preferable embodiment, and the scope of the present invention is not limited thereto. Accordingly, the substantial scope of the present invention is defined by the appended claims and their equivalents.

The ASCII text file “Sequence.txt” created on Mar. 2, 2022, having the size of 3,707 bytes, is incorporated by reference into the specification.

1-11. (canceled)

12. A method for preventing skin aging or regenerating the skin, the method comprising applying a composition containing fibroblast growth factor 17 (FGF 17) to the skin.

13. The method of claim 12, wherein the composition is for promoting proliferation ability of fibroblasts.

14. The method of claim 12, wherein the composition is for promoting migration ability of fibroblasts.

15. The method of claim 12, wherein the composition is used for application to the skin two or more times at intervals of 16 to 24 hours.

16. The method of claim 12, wherein the prevention of skin aging is any one or more uses selected from the group consisting of wrinkle alleviation, skin elasticity enhancement, skin regeneration enhancement, and skin shine improvement.

17. The method of claim 12, wherein the composition is for promoting collagen expression and for decreasing expressions of MMP1 (matrix metalloproteinase-1) and elastase.

18. A cosmetic method for skin regeneration, the method comprising applying a composition containing fibroblast growth factor 17 (FGF 17) to the skin.

19. The cosmetic method of claim 18, wherein the application to the skin is applying the composition containing FGF 17 two or more times.

20. The cosmetic method of claim 19, wherein the applying the composition two or more times to the skin is applying the composition at intervals of 16 to 24 hours to the skin.


SEQUENCE LISTING (18 sequences)

SEQ ID NO: 1 - DNA, 20 bp, Artificial Sequence
  beta-Actin (F) primer
agtcctgtggcatccacgaa

SEQ ID NO: 2 - DNA, 20 bp, Artificial Sequence
  beta-Actin (R) primer
gatccacacggagtacttgc

SEQ ID NO: 3 - DNA, 20 bp, Artificial Sequence
  Collagen I (F) primer
ccctcaaggtttccaaggac

SEQ ID NO: 4 - DNA, 20 bp, Artificial Sequence
  Collagen I (R) primer
accaggttcacccttcacac

SEQ ID NO: 5 - DNA, 21 bp, Artificial Sequence
  Collagen III (F) primer
tgaaaggacacagaggcttcg

SEQ ID NO: 6 - DNA, 20 bp, Artificial Sequence
  Collagen III (R) primer
gcaccattcttaccaggctc

SEQ ID NO: 7 - DNA, 20 bp, Artificial Sequence
  Elastin (F) primer
ctgcaaaggcagccaaatac

SEQ ID NO: 8 - DNA, 21 bp, Artificial Sequence
  Elastin (R) primer
caccaggaactaacccaaact

SEQ ID NO: 9 - DNA, 20 bp, Artificial Sequence
  GAPDH (F) primer
gtcggagtcaacggatttgg

SEQ ID NO: 10 - DNA, 21 bp, Artificial Sequence
  GAPDH (R) primer
gggtggaatcaattggaacat

SEQ ID NO: 11 - DNA, 20 bp, Artificial Sequence
  Collagen I (F) primer
tgcgatgacgtgatctgtga

SEQ ID NO: 12 - DNA, 20 bp, Artificial Sequence
  Collagen I (R) primer
ttggtcggtgggtgactctg

SEQ ID NO: 13 - DNA, 21 bp, Artificial Sequence
  collagen III (F) primer
tgaaaggacacagaggcttcg

SEQ ID NO: 14 - DNA, 20 bp, Artificial Sequence
  collagen III (R) primer
gcaccattcttaccaggctc

SEQ ID NO: 15 - DNA, 20 bp, Artificial Sequence
  Elastin (F) primer
ctgcaaaggcagccaaatac

SEQ ID NO: 16 - DNA, 21 bp, Artificial Sequence
  Elastin (R) primer
caccaggaactaacccaaact

SEQ ID NO: 17 - DNA, 18 bp, Artificial Sequence
  Elastage (F) primer
ttcctcgcctgtgtcctg

SEQ ID NO: 18 - DNA, 18 bp, Artificial Sequence
  Elastage (R) primer
ctgcagggacaccatgaa